(19) United States
(12) Patent Application Publication
(10) Pub. No.: US 2009/0105132 A1
(43) Pub. Date: Apr. 23, 2009

(54) Method for Enchancing Cyclodextrin Complexation

(75) Inventors: Pekka Jarho, Kuopio (FI); Tomi Jarvinen, Kuopio (FI); Kristiina Jarvinen, Kuopio (FI); Laura Matilainen, Kuopio (FI); Janne Mannila, Kuopio (FI)

Correspondence Address: BIRCH STEWART KOLASCH & BIRCH, PO BOX 747, FALLS CHURCH, VA, 22040-0747, US

(73) Assignee: PediPharm Oy, Kuopio (FI)

(21) Appl. No.: 12/224,778

(22) PCT Filed: Mar. 7, 2007
(86) PCT No.: PCT/FI2007/050122

§ 371 Date: Dec. 10, 2008

(30) Foreign Application Priority Data
Mar. 8, 2006 (FI) 20065154

(51) Int. Cl. A61K 38/13; C07K 14/00

(52) U.S. Cl. 514/11; 530/317

(57) ABSTRACT

The present invention is directed to a method for complexing a cyclodextrin with a cyclosporin, according to which method the complexation is carried out at a temperature of the most 15° C.

### FIELD OF THE INVENTION

The present invention describes a method for enhancing the complexation of cyclosporin with cyclodextrins (CDs). The improved complexation efficiency can be utilized especially for obtaining solid cyclosporin formulations exhibiting improved dissolution characteristics of cyclosporin. These CD containing solid formulations can be utilized especially in the oral administration of cyclosporin, including tablet and capsule formulations. In addition, the method can be utilized in formulations intended for pulmonary delivery of cyclosporin, such as in inhalation formulations. The enhancement method can, however, be utilized in all solid, semi-solid and liquid formulations containing cyclosporin and CDs comprising all drug administration routes.

### BACKGROUND OF THE INVENTION

The cyclosporins are a homologous group of biologically active oligopeptides. Cyclosporin A (CyA) is the best known member of this group, but also other cyclosporins have been identified. The cyclosporins share a cyclic peptide with 11 amino acid residues having different substituents or configuration of some of the amino acids. In the following, the term “cyclosporin” will be used to designate any individual member of the group of cyclosporins, or a mixture of such members.

Cyclosporin is an immunosuppressive drug and it is mainly used in the treatment of autoimmune diseases and to prevent the rejection of transplanted organs (Martindale 1999). From a pharmaceutical point of view the major problem of cyclosporin is its poor aqueous solubility and dissolution rate which lead to a low and variable oral bioavailability of cyclosporin. In order to improve the dissolution characteristics of cyclosporin, the drug has been formulated as a microemulsion (Sandimmune Neoral) which contains surfactant, lipophilic solvent, hydrophilic solvent and ethanol (Mueller et al. 1994).

Cyclodextrins (CDs) are cyclic oligosaecharides consisting of (α-1,4)-linked α-D -glucopyranose units, with a lipophilic central cavity and a hydrophilic outer surface (Frömming and Szejtli, 1994). CDs are able to form inclusion complexes with many drugs by taking up the whole drug, or more commonly, the lipophilic moiety of the molecule, into the cavity. The most abundant natural CDs are α-cyclodextrin (α-CD), β-cyclodextrin (β-CD) and γ-cyclodextrin (γ-CD), containing six, seven, and eight glucopyranose units, respectively. Since β-CD has limited aqueous solubility, numerous water-soluble β-CD derivatives have been synthesized, including hydroxypropyl-β-cyclodextrin (HP-β-CD), sulfobutylether-β-cyclodextrin (SBE-β-CD), branched CDs and methylated CDs, including dimethyl-β-cyclodextrin (DM-β-CD), trimethyl-β-cyclodextrin (TM-β-CD) and randomly methylated β-cyclodextrin (RM-β-CD).

In drug formulations, CDs have been used mainly to increase the aqueous solubility, stability and bioavailability of various drugs, food additives and cosmetic ingredients (Frömming and Szejtli, 1994). In addition, CDs can also be used to convert liquid compounds into microcrystalline powders, prevent drug-drug or drug-additive interactions, reduce gastro-intestinal or ocular irritation, and reduce or eliminate unpleasant taste and smell.

Studies dealing with the use of CDs with cyclosporins have shown that cyclosporin forms the most stable inclusion complex with natural α-CD, α-CD derivatives and methylated CDs. Kozo et al. (1991) showed that natural α-CD can be used to improve the aqueous solubility of cyclosporin A which can be utilized in ophthalmic delivery of CyA. Kozo et al. (1991) prepared aqueous solutions of CyA with natural α-CD and natural β-CD at room temperature and they showed that the highest CyA concentration (1.9 mg/ml) can be achieved with natural α-CD (2 g/10 ml). Kanai et al. (1989) studied the use of natural α-CD in the ophthalmic delivery of CyA. Kanai et al. (1989) report that the maximum solubility of CyA in a 8% solution of natural α-CD is 0.75 mg/ml. Kanai et al. (1989) do not report the temperature where the study was made. Miyake et al. (1999a) studied the effect of DM-β-CD on the oral absorption of CyA in rats. Miyake at al (1999a) prepared the CyA/DM-β-CD complex by using the kneading method. Miyake et al (1999a) have not reported the temperature at which the formulation was made. Miyake at al (1999b) have also studied the complexation of CyA with DM-α-CD and DM-β-CD by using the solubility method and spectrometric methods (MS, NMR). In this report the solubility studies were made at 25° C. and Miyake et al (1999b) report that a ˜2 mM (2.4 mg/ml) solubility of CyA was achieved with 50 mM DM-α-CD and DM-β-CD solutions. Okada et al. (1999) have studied the complexation properties of various branched CDs by using CyA as a model compound. In this study the solubility studies were performed at 30° C. and Okada et al. (1999) report that a ˜0,6 mM (0.7 mg/ml) solubility of CyA was achieved with the branched CDs (100 mM) studied. Ran et al. (2001) studied the effect HP-β-CD and α-CD on the aqueous solubility of CyA. Ran et al. (2001) report that a ˜1.2 mM (1.4 mg/ml) solubility of CyA can be achieved with 20% α-CD. Ran et al (2001) do not report the temperature where the study was made. Fukaya et al (2003) studied the effect of various CDs on the aqueous solubility of CyA and the use of a maltosyl-α-CD/CyA complex in the inhalation therapy of asthma. Fukaya et al. (2003) report that solubility studies were performed at room temperature and the highest solubility (27 mg/ml) of CyA was achieved with a 57% DM-β-CD-solution. With a 10% α-CD-solution Fukaya et al (1993) reported a 1.9 mg/ml solubility for CyA.

### SUMMARY OF INVENTION

The present invention is based on the discovery that the complexation of CDs with cyclosporins can be enhanced substantially by decreasing the temperature at which the complexation is carried out.

The present invention is thus directed to a method for complexing a cyclodextrin with a cyclosporin, according to which the complexation is carried out at a temperature of at the most 15° C.

The present invention is also directed to a method for enhancing the solubility and the dissolution rate of cyclosporin from a pharmaceutical preparation, the method comprising the steps of complexing the said cyclosporin with a cyclodextrin, whereby the complexation is carried out at a temperature of at the most 15° C., and forming the complex so obtained into a pharmaceutical preparation.

### DETAILED DESCRIPTION OF THE INVENTION

The present invention is based on the discovery that the complexation efficiency between cyclodextrin and cyclosporin can be enhanced by performing the complexation at a temperature below room or ambient temperature, that is at a temperature of at the most 15° C. It has been discovered that the complexation efficiency increases with decreasing temperature. Preferably the temperature is kept at the most at 10° C., and more preferably at the most at 5° C. The complexation reaction is suitably performed at as low a temperature as is practically possible, taking into account the complexation conditions, especially the complexation medium. Thus when carrying out the complexation in a water solution, the lower limit is dictated by the freezing point of the medium and thus a practical lower temperature limit is above the freezing temperature, such as at appr. 1° C. A suitable temperature range for complexation in an aqueous solution which is both practical for use while still providing sufficient complexation efficiency, is 1 to 10° C.

The cyclodextrin to be used according to the invention can be a natural cyclodextrin such as α-cyclodextrin (α-CD), β-cyclodextrin (β-CD) or γ-cyclodextrin (γ-CD), or it can be a modified cyclodextrin, such as hydroxypropyl-p-cyclodextrin (HP-β-CD), sulfobutylether-β-cyclodextrin (SBE-β-CD), a methylated CD, including dimethyl-β-cyclodextrin (DM-β-CD), triethyl-β-cyclodextrin (TM-β-CD) and randomly methylated β-cyclodextrin (RM-β-CD). Preferably the cyclodextrin is α-cyclodextrin or RM-β-cyclodextrin.

The cyclosporin can be any one belonging to the general group of cyclosporins. However, the preferred cyclosporin is cyclosporin A (CyA).

According to the method of the invention, cyclosporin is brought into contact with cyclodextrin in an amount to ensure maximum complexation efficiency between the two compounds to be completed. Thus the molar ratio used in the complexation reaction between cyclosporin and cyclodextrin preferably is in the range of 1:1 to 1:100. In this way maximum complexation efficiency between the two compounds can be achieved, for example enabling cyclosporin to be completed with cyclodextrin at a molar ratio, which depending on the temperature, can be as high as 1:11.

The complexation according to the invention can be carried out in a conventional manner, known to a person skilled in art, for example in solution, in a heterogenous state or in the solid state, or using supercritical conditions, such as supercritical carbon dioxide, using methods such as precipitation, freeze-drying, spray-drying, kneading, grinding, slurry-method, co-precipitation, and neutralization, and separating the said complex.

According to a preferred embodiment, the complexation is carried out in a complexation medium, such as a liquid medium. The liquid medium for use according to the invention can be water, an aqueous medium, for example a mixture of water and an organic solvent or a mixture of organic solvents, typically a water miscible solvent, or it can also be an organic solvent. Typical solvents which can come into use are for example lower alcohols, such as methanol or ethanol. The preferred liquid medium is water. If desired, or required, it is possible to modify the medium, for example to use pH-adjustment of the medium, using conventional pH regulating agents, such as acids or bases, or buffers, or any other adjuvant commonly used for this purpose.

According to such an embodiment, cyclosporin in a predetermined amount, and often in an excess as compared to its maximum solubility, can be added to a liquid medium containing a predetermined amount of cyclodextrin, for example a solution containing 10-20% by weight cyclodextrin. It is also possible to add both the cyclosporin and the cyclodextrin separately to a suitable liquid medium in order to carry out the complexation reaction. The mixture so obtained is stirred for a suitable period of time, while maintaining the liquid medium at the necessary low temperature. The stirring time can vary depending on the conditions, but a stirring time suitable for practical purposes is a few hours to a few days, such as from 2 or 3 hours to 6 days. After the complex formation is complete, the reaction mixture can be filtered, and the filtrate can be evaporated, suitably by freeze drying, for example under reduced pressure to give the desired complex.

The pharmaceutical preparation to be made from the complex obtained contains the said complex in a pharmaceutically acceptable amount together with pharmaceutically acceptable carriers, adjuvants or vehicles known in the art. The manufacture of such pharmaceutical formulations is well known in the art.

Although the invention is especially suitable for making solid oral dosage forms, such as tablets, capsules, or pills, the invention can be used for making any type of pharmaceutical formulations, such as semi-solid or liquid formulations both for oral as well as other forms of administration, such as parenteral administration, e.g. for injection, but also for pulmonary use, such as an inhalation formulation. All such preparations can be made using conventional methods of preparation known to the person skilled in the art.

The particular dosage to be administered to a patient, such as a mammalian, depends on the individual patient and his condition to be treated and can easily be determined by the person skilled in the art.

The present invention thus shows that the complexation of CDs with cyclosporin can be enhanced substantially by decreasing the temperature of the complexation medium. The present study shows that at room temperature a 1.2 mg/ml solution of CyA can be achieved with a 14% α-CD (Example 1) solution. The result is of the same order of magnitude compared to earlier studies (Kozo et al. 1991, Kanai and Alba 1989, Ran et al. 2001, Fukaya et al. 2003) which have been made at room temperature. However, Example 1 also shows that a 11.4 mg/ml solubility of CyA can be achieved if the complexation is done at +5° C. This result shows clearly that the complexation efficiency of cyclosporin with CDs increases at lower temperatures. Indeed, the best results were obtained at +1° C. where a 15.5 mg/ml solubility of CyA was achieved with a 14% α-CD solution. Similar results have been achieved in Example 2 by using RM-β-CD.

The improved complexation efficiency can be utilized especially in solid cyclosporin formulations containing CDs. As discussed before, CyA is commercially available as a microsuspension (Sandimmune Neoral) which contains doses of 25-100 mg of CyA. The present study shows that a α-CD/CyA complex which has been prepared at room temperature contains 1.2 mg CyA which has been complexed with 140 mg of CyA (Example 1). Thus, from this result it can be calculated that almost 6 g of α-CD would be needed to complex 50 mg of CyA, which is the average dose of the commercial microsuspension. This dose is far too big for oral applications. However, if the complexation is made at +1° C., it will contain 15.5 mg CyA which has been complexed with 140 mg of α-CD. From this result it can be calculated that 50 mg CyA can be complexed with 0.45 g of α-CD, which is a suitable amount for oral applications.

Improved complexation efficiency of cyclosporins with CDs can be utilized especially to improve the dissolution rate of cyclosporin from solid formulations. Example 3 shows the dissolution data of CyA from a solid CyA/α-CD complex which has been prepared at +1° C. The results show that α-CD complexation increases significantly the dissolution rate of CyA compared to pure CyA. Thus, the present invention improves substantially the usefulness of CDs especially in oral cyclosporin formulations. The invention can thus be used to improve the bioavailability and decrease the intra-individual variability of cyclosporin.

The following examples illustrate the invention without limiting the same in any way.

### EXAMPLE 1

In this example the effect of temperature on the complexation efficiency of CyA with α-CD has been described.

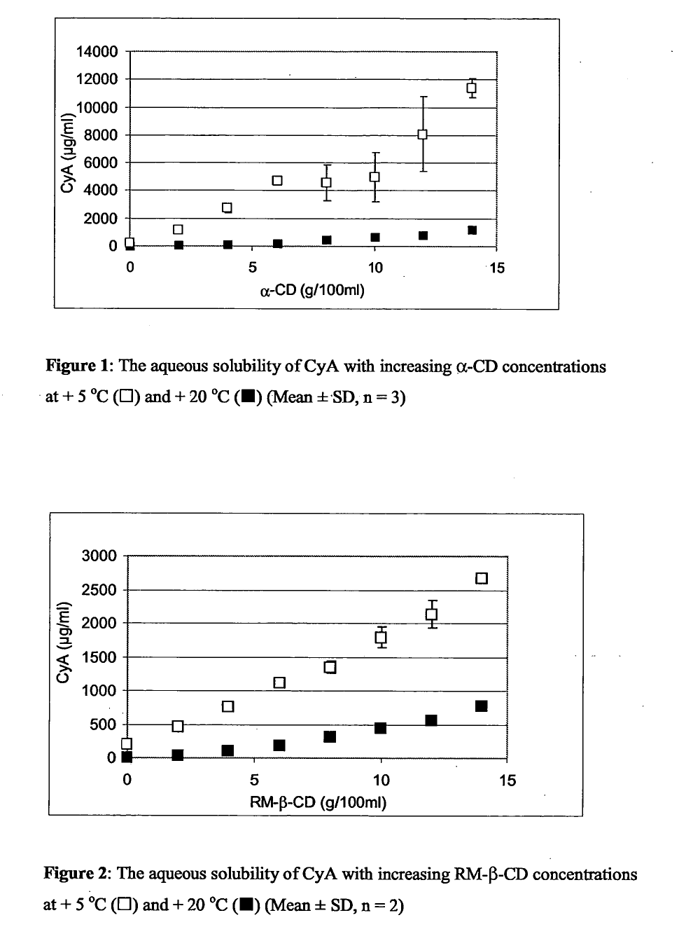

FIG. 1 shows the aqueous solubility of CyA with different concentrations of α-CD at +5 and +20° C. The results show clearly that the complexation efficiency of CyA with α-CD increases at lower temperatures. E.g., with a 14% α-CD solution the solubility of CyA is 11.4 mg/ml and 1.2 mg/ml at +5° C. and +20° C., respectively. Thus, after freeze-drying, the material which has been prepared at +5° C. contains 11.4 mg CyA which has been complexed with 140 mg of α-CD. Similarly, the material which has been prepared at +20° C. contains only 1.2 mg CyA which has been complexed with 140 mg of CyA.

The complexation studies were continued at +1° C. In this study a solubility of 15.5 mg/ml of CyA was achieved with 14% α-CD solution. Thus, after freeze-drying the material contained 15.5 mg CyA which was complexed with 140 mg of α-CD.

Thus according to this example the amount of cyclosporin that can be complexed to CD can be increased by a factor of more than 10 by decreasing the temperature below room or ambient temperature.

### EXAMPLE 2

In this example the effect of temperature on the complexation efficiency of CyA with RM-β-CD has been described.

FIG. 2 shows the aqueous solubility of CyA with different concentrations of RM-β-CD at +5 and +20° C. The result shows clearly that the complexation efficiency of CyA with RM-β-CD increases at lower temperatures. E.g. with a 14% RM-β-CD solution, the solubility of CyA is 2.7 mg/ml and 0.75 mg/ml at +5° C. and +20° C., respectively. Thus, after freeze-drying the material which has been prepared at +5° C. contains 2.7 mg CyA which has been complexed with 140 mg of RM-β-CD. Similarly, the material which has been prepared at +20° C. contains 0.75 mg CyA which has been complexed with 140 mg of CyA.

### EXAMPLE 3

In this example the effect of α-CD complexation on the dissolution rate of CyA has been described. The improved complexation efficiency between CyA and α-CD was utilized in the study.

The complex between CyA and α-CD was prepared as follows. An excess amount of CyA was added to a water solution which contained 14% α-CD. The suspension was shaken for six days at +1° C. After equilibration the suspension was filtered through 0.45 μm membrane filters and the resulting clear solution was freeze-dried. The HPLC analysis of the resulting material showed that 1 mg of CyA could be complexed with 9 mg of α-CD. All experiments were made in 6% α-CD solution to ensure the free dissolution of CyA.

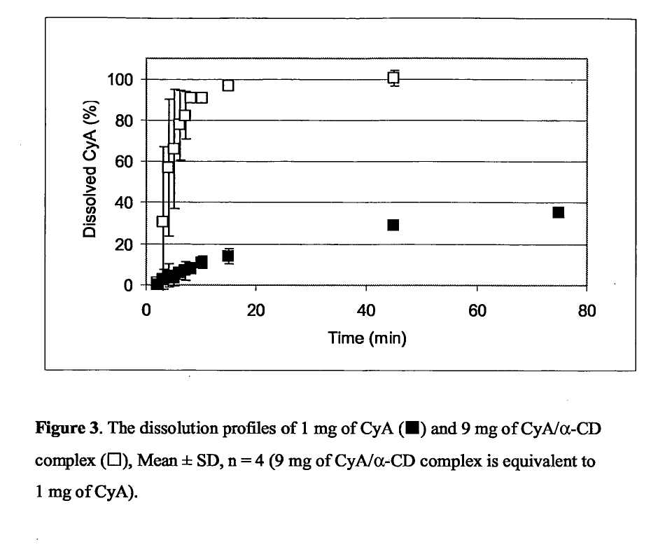

FIG. 3 shows the dissolution profile (dissolved CyA as a function of time) of CyA from a gelatine capsule containing 1 mg of pure CyA and a gelatine capsule containing 9 mg of CyA/α-CD complex (equivalent to 1 mg of CyA) prepared at +1° C. The results show clearly that the improved α-CD complexation increases significantly the dissolution rate of CyA.

### REFERENCES

Fukaya H, Limura A, Hoshiko K, Fuyumuro T, Noji S, Nabeshima T: A cyclosporin A/maltosyl-α-cyclodextrin complex for inhalation therapy of asthma. Eur. Respir. J. 22: 213-219, 2003.
Frömming K-H, Szejtli J: Cyclodextrins in pharmacy. Kluwer Academic Publishers, Dortrecht 1994.
Kanai A, Alba R M, Takano T, Kobayashi T, Nakajima A, Kurihara K, Yokoyama T, Fukami M: The effect on the cornea of alpha cyclodextrin vehicle for cyclosporin eyedrops. Transplantation Proceedings 21: 3150-3152, 1989.
Kozo K, Masaro M: Pharmaceutical composition containing cyclosporin in admixture with α-cyclodextrin. U.S. Pat. No. 5,051,402 (1991).
Martindale, The complete drug reference 32 edition, Pharmaceutical press 1999.
Miyake K, Arima H, Irie T, Hirayama F, Uekama K: Enhanced absorption of cyclosporin A by complexation with dimethyl-β-cyclodextrin in bile duct-cannylated and noncannylated rats. Biol. Pharm. Bull 22: 66-72, 1999a.
Miyake K, Hirayama F, Uekama K: Solubility and mass and nuclear magnetic resonance spectroscopic studies on interaction of cyclosporin A with dimethyl-α- and -β-cyclodextrins in aqueous solution J. Pharm. Sci. 88: 39-45, 1999b.
Mueller E A, Kovarik J M, van Bree J B, Tetzloff W, Grevel J, Kutz K: Improved dose linearity of cyclosporine pharmacokinetics from a microemulsion formulation. Pharm. Res. 11: 301-304, 1994.
Okada Y, Matsuda K, Hara K, Hamayasu K, Hashimoto H, Koizumi K: Properties and the inclusion behaviour of 6-O-α-D-galactosyl- and 6-O-α-D-mannosyl-cyclodextrins. Chem. Pharm. Bull 47: 1564-1568, 1999.
Ran Y, Zhao L, Xu Q, Yalkowsky S H: Solubilization of cyclosporin A. AAPS PharmSciTech 2 (1) article 2, 2001.

1. Method for complexing a cyclodextrin with a cyclosporin, characterized in that the complexation is carried out at a temperature of at the most 15° C.

2. The method according to claim 1, characterized in that the temperature is at the most 10° C., preferably at the most 5° C.

3. The method according to any preceding claim, characterized in that the complexation is carried out in a liquid medium.

4. The method according to claim 3, characterized in that the liquid medium is an aqueous medium.

5. The method according to claim 1, characterized in that the cyclosporin is cyclosporin A.

6. The method according to claim 1, characterized in that the cyclodextrin is α-cyclodextrin or RM-β-cyclodextrin.

7. The method according to claim 3, characterized in that the complexation is carried out by stirring for a few hours to a few days, optionally filtering the medium, and evaporating the filtrate to obtain the complex.

8. The method according to claim 1, characterized in that the molar ratio between the cyclosporin and cyclodextrin is 1:1 to 1:100.

9. The method according to claim 3, characterized in that cyclosporin in an excess amount is added to a cyclodextrin solution, preferably having a concentration of cyclodextrin of 10 to 20% by weight.

10. Method for enhancing the solubility and the dissolution rate of cyclosporin from a pharmaceutical preparation, the method comprising complexing the said cyclosporin with a cyclodextrin, whereby the complexation is carried out at a temperature of the most 15° C., and forming the complex obtained into a pharmaceutical preparation.

11. The method according to claim 10, the method comprising the step of forming the complex into a pharmaceutical preparation for oral or for pulmonary use.
